(19) United States
(12) Patent Application Publication
(10) Pub. No.: US 2009/0280160 A1
(43) Pub. Date: Nov. 12, 2009

(54) PHENYLEPHRINE PHARMACEUTICAL FORMULATIONS AND COMPOSITIONS FOR TRANSMUCOSAL ABSORPTION

(75) Inventors: David Monteith, Pittstown, NJ (US); John O'Mullane, Mountain Lakes, NJ (US); Joseph P. Reo, Lakeland, TN (US); Dennis Nelson, Memphis, TN (US); Jiansheng Wan, Warren, NJ (US); Xiaoming Chen, Westfield, NJ (US); Mohammed A. Kabir, Lakeland, TN (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering-Plough Healthcare Products, Inc.

(21) Appl. No.: 12/328,429

(22) Filed: Dec. 4, 2008

Related U.S. Application Data
(60) Provisional application No. 61/012,223, filed on Dec. 7, 2007.

(51) Int. Cl. A61K 9/70; A61K 9/00; A61K 9/14; A61K 9/12; A61K 31/137; C07C 215/54

(52) U.S. Cl. 424/443; 424/400; 424/489; 424/45; 514/653; 564/355

(57) ABSTRACT

Pharmaceutical compositions comprising phenylephrine or a pharmaceutically acceptable salt thereof and methods for administering the pharmaceutical compositions wherein the composition is formulated for systemic absorption of phenylephrine that avoids first pass metabolism. The compositions of the invention are formulated to be applied to oral mucosa of an animal to allow for enhanced systemic delivery of therapeutically active form of phenylephrine.

This application claims priority from U.S. provisional patent application Ser. No. 61/012,223 filed Dec. 7, 2007.

### BACKGROUND OF THE INVENTION

Identification or discussion of any reference in this section or any part of this specification shall not be construed as an admission that such reference is available as prior art to the present application.

Oral administration is the most preferred route for systemic pharmaceutical administration. However, oral administration of some pharmaceutical agents results in extensive pre-systemic metabolism of the agents as they undergo hepatic first pass metabolism and enzymatic metabolism within the gut wall. This extensive pre-systemic metabolism dramatically reduces the effective amount of pharmaceutical agent ultimately absorbed into the blood stream and available for therapeutic action. Transmucosal routes of drug delivery (i.e., the mucosal linings of the nasal, rectal, ocular, and oral cavity) offer advantages over oral administration of pharmaceutical agents that avoid the first pass effect and pre-systemic elimination within the gut wall, and speed absorption into the blood stream.

Phenylephrine undergoes extensive pre-systemic metabolism, with a majority of the metabolism taking place within the enterocytes of the gastrointestinal tract. (See, e.g., Ibrahim, K. E. et al., Journal of Pharmacy and Pharmacology 35, 144-147 (1983)). Phenylephrine is metabolized by Phase I and Phase II enzyme systems, mainly monoamine oxidase and suflotransferase, respectively. Ibrahim and coworkers measured the metabolism of phenylephrine after oral and inhalation administration and found four main metabolites were excreted in urine, unconjugated m-hydroxymandelic acid, sulfate conjugate of m-hydroxyphenylglycol, sulfate conjugate of phenylephrine and glucuronide conjugate of phenylephrine. The ratios of the phenyephrine metabolites differed depending on the route of administration, yet neither route demonstrated prolonged plasma levels of parent (unmetabolized) phenylephrine. Another study reported that oral administration of Comhist® tablets containing 10 or 20 mg of phenylephrine showed concentrations of parent phenylephrine in plasma were below the limit of quantitation of 2 ng/ml. (Gumbhir, K. An Investigation of Pharmacokinetics of Phenylephrine and its Metabolites in Humans. In Pharmaceutical Sciences, p. 216 (1993)).

U.S. patent application Ser. No. 11/756,881, filed Jun. 1, 2007, describe formulations that deliver phenylephrine and pharmaceutically acceptable salts thereof directly to the colon, avoiding pre-systemic metabolism. The application demonstrates that these formulations allow for systemic absorption of increased levels of parent phenylephrine compound resulting in demonstrable blood levels of parent phenylephrine for up to several hours.

Although the nasal, rectal and ocular mucosa offer certain advantages, the marginal patient acceptability renders them reserved for local applications rather than systemic drug administration. In particular, the potential irritation and the irreversible damage of the nasal cavity from chronic application make it less appealing as a method of administering several dosages as needed for effective systemic administration of phenylephrine. Alternatively, transdermal and oral mucosal delivery provide a highly acceptable administration route for chronic treatments. The oral mucosa is relatively permeable with a rich blood supply and demonstrates short recovery times after stress or damage. (Yajaman S., et al. J. Controlled. Release. 114:2006, 15-40; Rathbone, M. J. and Hadgraft, J., Int. J. Pharm., 74:9-24, 1991; Squier, C. A., Crit. Rev. Oral Biol. Med., 2:13-32, 1991. 15. Squier, C). The virtual lack of Langerhans cells makes the oral mucosa tolerant to potential allergens. (Harris, D. and Robinson, J. R., J. Pharm. Sci., 81:1-10, 1992) Oral transmucosal drug delivery also bypasses liver first pass metabolism and avoids pre-systemic elimination in the gastrointestinal tract.

Thus, a composition that would allow for substantial systemic administration of unmetabolized phenylephrine would be useful. Further, a composition that allowed for prolonged administration of unmetabolized phenylephrine would be useful. Further orally administered phenylephrine compositions which avoid the metabolic issues associated with oral systemic administration would be useful.

These and other objectives are provided by the invention described and claimed herein. All references cited herein are hereby incorporated in their entirety into the subject application.

### SUMMARY OF THE INVENTION

This invention provides a pharmaceutical composition comprising phenylephrine or a pharmaceutically acceptable salt thereof, wherein the composition is formulated to be applied to oral mucosa to allow for enhanced systemic absorption of therapeutically active form of phenylephrine.

This invention further provides a pharmaceutical composition suitable for sublingual systemic administration of phenylephrine or a pharmaceutically acceptable salt thereof, wherein the composition allows for systemic absorption of phenylephrine from the floor of the mouth.

This invention also provides a pharmaceutical composition suitable for buccal systemic administration of phenylephrine or a pharmaceutically acceptable salt thereof, wherein the composition allows for absorption of phenylephrine from the buccal mucosa.

This invention also provides a method of systemically administering phenylephrine which comprises contacting oral mucosa with a pharmaceutical composition comprising phenylephrine or a pharmaceutically acceptable salt thereof, wherein the composition allows for release of phenylephrine to oral mucosa.

This invention further provides a dissolvable composition comprising phenylephrine distributed within an aqueous soluble base material, wherein the composition is provided as a strip for inter-oral administration of phenylephrine to the mucus membranes of the mouth of a human or animal subject.

This invention also provides a bioerodible, water-soluble, carrier device comprising a non-bioadhesive backing layer, a bioadhesive layer and a composition comprising phenylephrine or a pharmaceutically acceptable salt thereof, wherein the bioadhesive layer is formulated to adhere to a mucosal surface of a mammal and provides sustained delivery of the composition.

This invention further provides a composition for buccal or sublingual application comprising a distribution of multilayer microparticles in a base, wherein phenylephrine or a pharmaceutically acceptable salt thereof is adsorbed within the layers of the microparticles so as to be progressively released over time to the buccal or sublingual mucosa.

This invention also provides a drug delivery device adapted for application sublingually of the oral cavity for fast release thereon of a composition comprising phenylephrine or a pharmaceutically acceptable salt thereof, said device comprising a body having the composition distributed therein and having a size and shape suitable for sublingual application

This invention also provides a pharmaceutical formulation adapted for application and adherence to the mucosa of the oral cavity for sustained release thereon of a composition comprising phenylephrine or a pharmaceutically acceptable salt thereof wherein the composition is in the form of a liquid or semisolid.

### BRIEF DESCRIPTION OF THE FIGURES

FIGS. 1A and 1B: graphs showing calcium flux studies demonstrating that phenylephrine (▪) but not 3-hydroxymandelic acid (♦) increases intracellular calcium in α₁ₐ and α_{1b} expressing CHO cells.

FIGS. 2A and 2B: graphs showing receptor binding studies demonstrating that phenylephrine (▪) but not 3-hydroxymandelic acid (♦) inhibits binding of ³H-prazosin to α₁ₐ and α_{1b} CHO cell membranes.

FIGS. 3A, 3B, and 3C: graphs showing receptor binding studies demonstrating that phenylephrine (▪) but not 3-hydroxymandelic acid (▴ (3A, 3B), ♦ (3C)) stimulates [³⁵S]-GTPγS binding to α₂ₐ and α_{2b} and α_{2c} CHO cell membranes.

FIGS. 4A, 4B, and 4C: graphs showing receptor binding studies demonstrating that phenylephrine (▪) but not 3-hydroxymandelic acid (▴) inhibits [³H]-UK14304 binding to α₂ₐ and α_{2b} and α_{2c} CHO cell membranes.

FIGS. 5A and 5B: graphs showing calcium flux studies demonstrating that phenylephrine sulfate (▴) induces minimal intracellular calcium increases in α₁ₐ and α_{1b} expressing CHO cells. (▪=PE; =Theoretical 0.1% PE)

FIGS. 6A and 6B: graphs showing receptor binding studies demonstrating that phenylephrine (▪) but not PE sulfate (▴) inhibits binding of ³H-prazosin to α₁ₐ and α_{1b} CHO cell membranes. (=Theoretical 0.1% PE)

FIGS. 7A, 7B, and 7C: graphs showing receptor binding studies demonstrating that phenylephrine (▪) but not PE sulfate (▴) stimulates [³⁵S]-GTPγS binding to α₂ₐ and α_{2b} and α_{2c} CHO cell membranes. (=Theoretical 0.1% PE)

FIGS. 8A, 8B, and 8C: graphs showing receptor binding studies demonstrating that phenylephrine (▪) but not PE sulfate (▴) inhibits [³H]-UK14304 binding to α₂ₐ and α_{2b} and α_{2c} CHO cell membranes. (=Theoretical 0.1% PE)

FIGS. 9A and 9B: graphs showing calcium flux studies demonstrating that PE glucuronide (▴) induces intracellular calcium increases in α₁ₐ and α_{1b} expressing CHO cells consistent with level of contaminating phenylephrine. (▪=PE; =Theoretical 0.28% PE)

FIGS. 10A and 10B: graphs showing receptor binding studies demonstrating that phenylephrine (▪) but not PE glucuronide (▴) (batch 2) inhibits binding of ³H-prazosin to α₁ₐ and α_{1b} receptors (CHO cell membranes).

FIGS. 11A, 11B, 11C: graphs showing receptor binding studies demonstrating that phenylephrine (▪) but not PE glucuronide (▾) (batch 2) stimulates [³⁵S]-GTPγS binding to α₂ₐ and α_{2b} and α_{2c} CHO cell membranes.

FIGS. 12A, 12B, and 12C: graphs showing receptor binding studies demonstrating that PE glucuronide (▴) weakly inhibits binding of [³H]-UK14304 to α₂ₐ, α_{2b}, and α_{2c} receptors (CHO cell membranes) consistent with level of contaminating phenylephrine. (▪=PE; =Theoretical 0.28% PE)

### DETAILED DESCRIPTION

The subject invention provides a pharmaceutical composition comprising phenylephrine or a pharmaceutically acceptable salt thereof, wherein the composition is formulated for enhanced systemic absorption of phenylephrine that avoids first pass metabolism. In certain embodiments, the compositions of the invention are formulated to be applied to oral mucosa of an animal, human or otherwise, to allow for enhanced systemic delivery of therapeutically active form of phenylephrine, and thus optimize systemic exposure of a therapeutically active form of phenylephrine, by-passing pre-systemic metabolism.

As used herein a pharmaceutically acceptable salt of phenylephrine includes but is not limited to phenylephrine hydrochloride, phenylephrine bitartrate, phenylephrine tannate, etc. In one preferred embodiment, the pharmaceutically acceptable salt of phenylephrine is phenylephrine hydrochloride.

The term “unmetabolized phenylephrine” means Phenylephrine that has not been biotransformed by Phase I or Phase II enzymes systems, or any other enzyme system, into a new chemical entity since entering the body of a subject except for the release of free base, i.e. Phenylephrine that has not been conjugated by a sulfotransferase or a UDP-glucuronsyltransferase enzymes, or chemically altered by any enzyme system in the body of a subject, including enzyme systems of microbial organisms. Unmetabolized phenylephrine exhibits therapeutic activity(ies). “Unmetabolized phenylephrine” does not include phenylephrine that was at one time inactivated by conjugation but was later unconjugated and is not therapeutically active. The term “enhanced systemic absorption of therapeutically active form of phenylephrine” as used herein refers to the increased amount of therapeutically active chemical form of the administered phenylephrine, i.e., unmetabolized phenylephrine, absorbed into the systemic circulation and distributed to the body tissues, often characterized as area under the plasma concentration versus time curve, as compared to non-oral mucosal drug delivery forms.

The term “pre-systemic modification” as used herein in connection with phenylephrine means modification of phenylephrine before phenylephrine is taken Lip into the bloodstream and thus into the plasma. Pre-systemic modification excludes modification of phenylephrine by the liver or within the bloodstream.

As used here, the term “systemic oral mucosal delivery” means administration to mucosal membranes within the oral cavity for systemic uptake. The compositions and methods of the invention described herein are designed to take advantage of administration to the non-keratinized epithelia, such as found in the mucosa of the soft palate, the floor of the mouth and the buccal mucosa which are considerably more permeable to water and other small molecules compared to keratinized epithelia. In particular, oral mucosal delivery is meant to include sublingual delivery, which is systemic delivery of drugs through the mucosal membranes lining the floor of the mouth, as well as buccal delivery, which is drug administration through the mucosal membranes lining the cheeks (buccal mucosa). The permeability of oral mucosae found to be in between that of the epidermis and intestinal mucosa. In general, the permeabilities of the oral mucosae decrease from the sublingual to buccal, and buccal to palatal region. The sublingual mucosa is comparatively more permeable and rapid absorption leads to acceptable bioavailabilities of many drugs, and is convenient, accessible, and generally well accepted (Harris, D. and Robinson, J. R., Drug delivery via the mucous membranes of the oral cavity, J. Pharm. Sci., 81:1-10, 1992). The subject invention contemplates administration of phenylephrine to these regions of the oral mucosa that will allow for similar systemic uptake of parent phenylephrine.

A “dosage” or “dose” as used herein means the amount of a pharmaceutical composition comprising therapeutically active agent(s) administered at a time. “Dosage” or “dose” includes administration of one or more units of pharmaceutical composition administered at the same time.

“AUC” as used herein means, for any given drug, the “area under the concentration-time curve” from dosing or activation of the drug to a time point, calculated by the trapezoidal rule. AUC is a parameter showing the cumulative plasma concentration of a drug over time, and is an indicator of the total amount and availability of a drug in the plasma. “AUC₀₋ₜ” is defined as AUC for any value of time (t) up to 24 hours. In a preferred embodiment, t is 24 hours (referred to herein as AUC₀₋₂₄). “AUC_{0-∞}.” is defined as calculated AUC extrapolated to infinity. AUC_{0-∞} is calculated as equal to AUC₀₋ₜ t+Ct/λz, wherein Ct is the concentration at 24 hours and λz is the terminal or elimination rate constant. Terminal or elimination rate constant λz is determined from the slope of the drug concentration-time curve using linear regression on terminal data points of the curve. “Relative AUC₀₋ₜ” is defined as the percentage of the AUC₀₋ₜ value of unconjugated phenylephrine relative to the AUC₀₋ₜ value for the total phenylephrine in the plasma of the subject from a dosing regimen.

### Pharmaceutical Compositions

The compositions of the invention can take on any of several forms suitable for oral administration of pharmaceutical compositions including liquid, solid or semi-solid.

Liquid forms can be those suitable for spraying from a pump spray or pressurized spray device such as an aerosol spray. Liquids can also be delivered to the oral mucosa from a solid carrier such as a capsule that can be opened and its contents emptied into the mouth. For example, U.S. Pat. Nos. 6,676,931 6,969,508, 6,767,925 disclose liquid formulations that deliver an active agent to the mouth for absorption through the oral mucosa, for example by spraying.

Solid forms encompass all forms that are devised to be inserted into the mouth and either masticated or allowed to dissolve to release a pharmaceutical agent and include, but are not limited to, tablets, capsules, gums, films, lozenges, discs, spheres, and microspheres. For example, U.S. Patent Nos. RE 33,093 and 6,072,100, and 6375963 describe bioadhesive hot-melt extruded films for intra-oral drug delivery and the processing thereof. U.S. Pat. No. 6,596,298 describes orally dissolving films with no mucoadhesive properties. U.S. Pat. No. 6,284,264 describes mucoadhesive orally dissolving films. U.S. Pat. No. 4,755,389 discloses hard gelatin capsule filled with a chewable composition containing an ingredient for buccal absorption. U.S. Pat. No. 5,437,872 describes pharmaceutical tablet and lozenge forms providing controlled and sustained release of pharmaceutical agents. Such forms can also include forms referred to as fast dissolve, fast melt, and flash melt solid forms. For example U.S. Pat. No. 6,723,348 describes fast dissolving tablets that disintegrate in the buccal cavity upon contact with saliva by formation of an easy-to-swallow suspension. U.S. Pat. Nos. 5,464,632, 6,106,861, and 6,656,492 and PCT Published applications WO 00/27357 and WO00/51568 describe fast dissolving tablet formulations where the active ingredient is in the form of orally disintegratable tablet containing coated microcrystals or coated microgranules.

Semi-solid forms include, but are not limited to, chewing gums, viscous liquids, ointments, gels and hydrogel systems. For example, U.S. Pat. Nos. 7,078,052, 6,773,716 and 6,558,692 disclose pharmaceutical chewing gum formulations for delivering active agents to the oral mucosa.

In certain embodiments the compositions of the invention may also comprise multilayered forms containing a combination of fast dissolve and slow dissolve layers. As used herein the term multilayered is not limited to discrete layers of materials but can also include mixtures of particles having slow dissolve and fast dissolve properties.

In certain embodiments of the invention, the composition is formulated to allow for immediate systemic absorption of phenylephrine. In additional embodiments of the invention, the composition is formulated to allow for sustained systemic absorption of phenylephrine. In additional embodiments of the invention the composition is formulated to allow for both an immediate systemic absorption and a sustained systemic absorption of phenylephrine.

In certain embodiments the composition is suitable for sublingual administration such that the composition allows for systemic absorption of phenylephrine from the floor of the mouth.

In certain embodiments the composition is suitable for buccal administration such that the composition allows for absorption of phenylephrine from the buccal mucosa. Buccal mucosa has excellent accessibility with the direct access to the systemic circulation through the internal jugular vein which would bypass phenylephrine from the presystemic metabolism. Certain embodiments of the invention suitable for buccal administration can include matrix tablets and films. In certain embodiments the compositions of the invention suitable for buccal administration will have at least one of the followings properties: (i) adhere to the buccal mucosa for few minutes to several hours; (ii) release phenylephrine by either or both of immediate burst or controlled release; (iii) release phenylephrine in an unidirectional manner directly to the mucosa or all directions; (iv) facilitate drug absorption through buccal mucosa; (vi) adapted to not interfere with normal function such as talking or drinking.

In certain embodiments the composition of the invention can comprise a dissolvable composition comprising phenylephrine distributed within an aqueous soluble base material, wherein the composition is provided as a strip for inter-oral administration of phenylephrine to the mucus membranes of the mouth of a human or animal subject. In certain embodiments, the dissolvable composition can comprise a base material comprising a carrier which is conformed as a strip to serve as a delivery system for a measured dose of phenylephrine. In certain embodiments, the strip can be a film impregnated with, coated with or otherwise carry phenylephrine to enable the distribution of the phenylephrine to the oral cavity. The films generally comprise one or more water-soluble or water-swellable thermoplastic polymers such as hydroxypropylcellulose, polyethylene oxide, homopolymers and copolymers of carboxymethyl cellulose, hydroxyethyl cellulose, hydroxymethyl cellulose) with or without a plasticizer. The strip/film can have a thickness suitable for oral administration to a subject, typically of from about 20 microns to about 250 microns.

In certain embodiments, the composition may comprise part or all of the phenylephrine or pharmaceutically acceptable salt thereof encapsulated within encapsulation structures. The encapsulation structures may be selected to provide adhesion to the mucous membranes of the oral cavity and/or be adapted to release the phenylephrine slowly over time. In certain embodiments, the encapsulation structures may comprise multilamellar microparticles.

In certain embodiments, the composition of the invention can comprise a bioerodible, water-soluble, carrier device comprising a non-bioadhesive backing layer, a bioadhesive layer and a composition comprising phenylephrine or a pharmaceutically acceptable salt thereof. In certain embodiments, the bioadhesive layer may be formulated to adhere to an oral mucosal surface to enable sustained delivery of the composition. In certain embodiments the carrier device may further comprise a fluid carrier suitable for administration to a mucosal surface of a mammal. The fluid carrier may comprise one or more of such materials as acetic acid, acetone, anisole, 1-butanol, 2-butanol, butyl acetate, tert-butylmethyl ether, cumene, dimethyl sulfoxide, ethanol, ethyl acetate, ethyl ether, methanol, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, or tetrahydrofuran. In certain embodiments, the carrier device may further comprises a polymeric or nonpolymeric hydrophilic agent, such as polyethylene glycol.

In certain embodiments, the compositions of the invention can comprise a non-bioadhesive backing layer such as a pharmaceutically acceptable, film-forming, water-soluble polymer. Examples of pharmaceutically acceptable, film-forming, water-soluble polymer include, but are not limited to, hydroxyethyl cellulose, hydroxypropyl cellulose, hydroxypropylmethyl cellulose, hydroxyethylmethyl cellulose, polyvinyl alcohol, polyethylene glycol, polyethylene oxide, ethylene oxide-propylene oxide co-polymers, and combinations thereof.

In certain embodiments, the composition of the invention may comprise a distribution of multilayer microparticles in a base, wherein phenylephrine or a pharmaceutically acceptable salt thereof is adsorbed within the layers of the microparticles so as to be progressively released over time to the buccal or sublingual mucosa. Compositions containing such microparticles can be administered by various means, such as film, gel, capsule, tablet, aerosolized or otherwise pressurized spray, non-pressurized pump spray, mousse or drench, etc. In certain embodiments, the distribution of multilayer microparticles is in the form of a soluble solid or gel base, the base material being formulated to dissolve within the mouth and liberate the microparticles to allow for contact of the microparticles with the mucous membranes of the oral cavity. In certain embodiments, multilayer microparticles are in the range 0.1-10 microns. In certain embodiments, the microparticles may comprise polar structures with a positive surface charge to allow for adhesion to mucosal surfaces. U.S. Pat. No. 6,861,066 describes the use of high shear rates, such as with a microfluidizer, to produce uniform submicron particle and droplet sizes of chemical or particulate substances.

In certain embodiments, the compositions of the invention may provide for a sustained release of phenylephrine to provide a measurable blood levels of parent (unmetabolized) phenylephrine in a subject for a sustained period of time, wherein the period of time is at least about 5, 10, 15, 30, or 45 minutes, or at least about 1, 2, 3, 4, 5, 6, 7, 8, 9, 10, 11, 12, 13, 14, 15, 16, 17, 18, 19, 20, 21, 22, 23, or 24 hours.

In certain embodiments, the compositions of the invention may contain additional therapeutic agents in addition to phenylephrine. The additional therapeutic agent may be a decongestant including anti-histamine, an anti-pyretic, a non-steroidal anti-inflammatory, or any other therapeutic agent or combination of two or more of such agents to assist alleviation of the symptoms of a cold, a seasonal or non-seasonal allergy, hay fever, or sinus problems. In a preferred embodiment, the pharmaceutical compositions include an antihistamine. Antihistamines can be of H1 or H2 antagonists or other types of histamine release inhibitors. The H1 antagonists can be sedating or non-sedating, such as diphenhydramine, chlorpheniramine, tripelennamine, promethazine, clemastine, doxylamine, astemizole, terfenadine, and loratadine, among others. Examples of H2 antagonists include, but are not limited to, cimetidine, famotidine, nizatidine, and ranitidine. Examples of histamine-release inhibitors include cromolyn. Long-acting antihistamines selected from one or more of the group consisting of loratadine, desloratadine, azatidine, fexofenadine, terfenadine, cetirizine, astemizole, and levocabastine, or their pharmaceutically acceptable salts are suitable for the pharmaceutical compositions of the invention.

Preferred antihistamines include loratadine and desloratadine. Loratadine is disclosed in U.S. Pat. No. 4,282,233 as a non-sedating antihistamine useful, for example, in alleviation of seasonal allergic rhinitis symptoms such as sneezing and itching. The active metabolite of loratadine is desloratadine, which has a half-life (t_{1/2}) of approximately 15 to 19 hours. U.S. Pat. No. 5,595,997 discloses methods and compositions for treating seasonal allergic rhinitis symptoms using desloratadine. Loratadine and desloratadine are available in the form of conventional tablets that release the active agent in a conventional manner. An exemplary formulation releases loratadine by the processes of disintegration and dissolution such that loratadine begins to elicit its antihistaminic effect within 1 to 3 hours and the effect lasts in excess of 24 hours. Due to the long half life of loratadine compared to phenylephrine, the loratadine in the formulation according to the present invention is preferably available for immediate release. For example, loratadine or desloratadine may be present in solution in the carrier liquid of a liquid core or incorporated into the top coating of the product. Other antihistamines are also useful for the practice of the instant invention. Azatadine is disclosed in Belgian Patent No. 647, 043 and in corresponding U.S. Pat. Nos. 3,326,924 and 3,419,565. The elimination half-life is reported to be 9-12 hours. Terfenadine and fexofenadine are disclosed in U.S. Pat. No. 3,878,217 and have a duration of action of 12 to 24 hours, and greater than 24 hours, respectively. Cetirizine is disclosed in U.S. Pat. No. 4,525,358 and is reported to have a duration of action of 12 to 24 hours. Astemizole is disclosed in U.S. Pat. No. 4,219,559 and is reported to have a duration of action greater than 24 hours. Levocabastine is disclosed in U.S. Pat. No. 4,369,184 and is reported to have a duration of action of 16 to 24 hours. The dosage of antihistamine such as loratadine or desloratadine may be present in different concentrations such as 1-20 mg; preferably 2.5 mg, 5 mg, or 10 mg.

Suitable anti-inflammatory and/or antipyretic agents useful for the present compositions may be: a non-steroidal anti-inflammatory (NSAIDs), aminoarylcarboxylic acid derivatives such as enfenamic acid, etofenamate, flufenamic acid, isonixin, meclofenamic acid, mefanamic acid, niflumic acid, talniflumate, terofenamate and tolfenamic acid; arylacetic acid derivatives such as acemetacin, alclofenac, amfenac, bufexamac, cinmetacin, clopirac, diclofenac sodium, etodolac, felbinac, fenclofenac, fenclorac, fenclozic acid, fentiazac, glucametacin, ibufenac, indomethacin, isofezolac, isoxepac, lonazolac, metiazinic acid, oxametacine, proglumetacin, sulindac, tiarammide, tolmetin and zomepirac; arylbijtyric acid derivatives such as bumadizon, butibufen, fenbufen and xenbucin; arylcarboxylic acids such as clidanac, ketorolac and tinoridine; arylpropionic acid derivatives such as alminoprofen, benoxaprofen, bucloxic acid; carprofen, fenoprofen, flunoxaprofen, flurbiprofen, ibuprofen, ibuproxam, indoprofen, ketoprofen, loxoprofen, miroprofen, naproxen, oxaprozin, piketoprofen, pirprofen, pranoprofen, protizinic acid, suprofen and tiaprofenic acid; pyrazoles such as difenamizole and epirizole; pyrazolones such as apazone, benzpiperylon, feprazone, mofebutazone, morazone, oxyphenbutazone, phenybutazone, pipebuzone, propyphenazone, ramifenazone, suxibuzone and thiazolinobutazone; salicylic acid derivatives such as acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamine o-acetic acid, salicylsulfuric acid, salsalate and sulfasalazine; thiazinecarboxamides such as droxicam, isoxicam, piroxicam and tenoxicam; others such as ÿ-acetamidocaproic acid, s-adenosylmethionine, 3-amino-4-hydroxybutyric acid, amixetrine, bendazac, benzydamine, bucolome, difenpiramide, ditazol, emorfazone, guaiazulene, nabumetone, nimesulide, orgotein, oxaceprol, paranyline, perisoxal, pifoxime, proquazone, proxazole and tenidap; and pharmaceutically acceptable salts thereof; and other analgesics, such as acetaminophen. The dosage of analgesic and/or antipyretic such as aspirin, acetaminophen, etc. will be known to those skilled in the art and can be in the range of 80 mg to 250 mg. The dosage of NSAID will be known to those skilled in the art and can be in the range of 80 mg to 500 mg.

Certain embodiments of the compositions of the invention are designed to release phenylephrine unidirectionally targeting the oral mucosa. Additional embodiments of the compositions of the invention are designed to release phenylephrine multidirectionally directly to the mucosa and into the saliva. Certain embodiments of the compositions of the invention may also contain a pharmaceutically acceptable bioadhesive or mucoadhesive additive to promote retention of the composition in the oral cavity for a period of time to allow for sustained release of phenylephrine. Examples of pharmaceutically acceptable bioadhesives and mucoadhesives are known in the art and include, but are not limited to, cellulose derivatives such as hydroxypropyl cellulose, and others as described in U.S. Pat. No. 4,940,587. In certain embodiments, the bioadhesive layer can be water-soluble or non-water soluble. Certain water soluble bioadhesive layers include film forming water-soluble polymers and bioadhesive polymers. Examples of film forming water soluble polymers include, but are not limited to, hydroxyethyl cellulose, hydroxypropyl cellulose, hydroxypropylmethyl cellulose, hydroxyethylmethyl cellulose, and combinations thereof. In certain embodiments, the film forming water soluble polymer of the bioadhesive layer is crosslinked or plasticized. Examples of bioadhesive polymers include, but are not limited to, polyacrylic acid, sodium carboxymethyl cellulose or polyvinylpyrrolidone and combinations thereof. In certain embodiments, polyacrylic acid can be fully or partially crosslinked. Examples of mucoadhesives include gels, pastes, macromolecules, polymers, and oligomers, and mixtures thereof that can adhere to a subject's mucous membrane for a period of time sufficient to deliver the active agent such as described in U.S. Pat. No. 6,509,028.

In certain embodiments the compositions of the invention comprise at least one or a combination of biodegradable polymers to form a matrix with the phenylephrine or pharmaceutically acceptable salt thereof such that the matrix would provide an instant phenylephrine release upon contact with oral mucous without taking any water. In certain embodiments, the matrix can be in the form of a film or lattice comprising the biodegradable polymers. Such polymers are known in the art and can be selected from non-limiting examples including gelatin, dextran, dextrin, alginates (i.e., sodium alginate), hydroxypropyl methylcellulose (HPMC), hydroxypropylcellulose, carboxymethylcellulose or its salt, polyvinyl alcohol, polyvinylpyrrolidine, sucrose or other compressible sugars, dextrose, dextrate, maltodextrine, starch, modified starch, microcrystalline cellulose, silidified microcrystalline cellulose, polyethylene glycols, lactose or with other pharmaceutically acceptable carrier materials. In certain embodiments, the compositions of the invention may also contain a pharmaceutical wax could be added for better performance.

The compositions of the invention may optionally comprise a penetration enhancer. Examples of penetration enhancers are: salicylates such as sodium salicylate, 3-methoxysalicylate, 5-methoxysalicylate and homovanilate; bile acids such as taurocholic, tauorodeoxycholic, deoxycholic, cholic, glycholic, lithocholate, chenodeoxycholic, ursodeoxycholic, ursocholic, dehydrocholic, fusidic, etc.; non-ionic surfactants such as polyoxyethylene ethers (e.g. Brij 36T®, Brij 52®, Brij 56®, Brij 765, Brij 965, Texaphore® A6, Texaphor® A14, Texaphor® A60 etc.), p-t-octyl phenol polyoxyethylenes (Triton® X-45, Triton® X-100, Triton® X-114, Triton® X-305 etc.) nonylphenoxypoloxyethylenes (e.g. Igepal® CO series), polyoxyethylene sorbitan esters (e.g. Tween®-20, Tween®-80 etc.); anionic surfactants such as dioctyl sodium sulfosuccinate; lyso-phospholipids such as lysolecithin and lysophosphatidylethanolamine; acylcarnitines, acylcholines and acyl amino acids such as lauroylcarnitine, myristoylcarnitine, palmitoylcarnitine, lauroylcholine, myristoylcholine, palmitoylcholine, hexadecyllysine, N-acylphenylalanine, N-acylglycine etc.; water soluble phospholipids; medium-chain glycerides which are mixtures of mono-, di- and triglycerides comprising medium-chain-length fatty acids (caprylic, capric and lauric acids); ethylene-diaminetetraacetic acid (EDTA); cationic surfactants such as cetylpyridinium chloride; fatty acid derivatives of polyethylene glycol such as Labrasol®, Labrafac®, etc.; and alkylsaccharides such as lauryl maltoside, lauroyl sucrose, myristoyl sucrose, and palmitoyl sucrose.

Certain embodiments of the compositions of the invention may comprise one or more solubilizing agents with phenylephrine or other active agents to promote rapid dissolution in aqueous media. Suitable solubilizing agents include wetting agents such as polysorbates and poloxamers, non-ionic and ionic surfactants, food acids and bases (e.g. sodium bicarbonate), and alcohols, and buffer salts for pH control. Suitable acids include, but are not limited to, acetic acid, ascorbic acid, citric acid, and hydrochloric acid.

Certain embodiments of the compositions of the invention may comprise buffering materials to assist in absorption of pharmaceutically active ingredients. Certain embodiments of buffered formulations may include sodium carbonate, sodium phosphate, calcium carbonate, magnesium hydroxide, magnesium carbonate, aluminum hydroxide, or combinations thereof and other similar substances known to those skilled in the art. Certain embodiments of the invention will optionally contain taste masking agents, such as flavors and/or sweeteners. The compositions may further comprise one or more lubricating and/or moisturising oils, including but not limited to hyaluronic acid or sodium hyaluronate, glycerol, calendula officinalis flower extract or glycerin extract, guar hydroxypropyltrimonium chloride, xanthan gum, cellulose gum, sodium chloride, olive oil, sunflower oil, almond oil, sesame oil, aloe vera, aloe barbadensis, and combinations thereof.

### General Process for Manufacturing the Formulations

Another aspect of the invention are the processes of manufacturing the formulations described above. The solid formulations are prepared using methods generally known in the art to prepare orally delivered, single layer and multiple-layered dosage forms. See, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980). Stability and degradation analyses can be performed according to the International Conference on Harmonization (ICH) standards as described in “Impurities in New Drug Products” guidelines to simulate two or more years of shelf life. For example, stability testing can be performed at 40 degrees Celsius/75% relative humidity for a 3-month period. Standard pharmaceutical storage conditions are known in the art. Compositions according to the invention can be assayed to meet all ICH guidelines for active pharmaceutical assay with degradant levels which are below reporting limits, preferably below identification limits, and most preferably below qualification limits. The compositions of the invention can be packaged maintain stability of the product. Preferred packaging methods include strip lamination in a foil-like material or packaging in blisters using a foil or teflon-like material.

### Methods of Treatment and Administration

The methods of the invention are directed to administration of the pharmaceutical compositions for temporary relief of congestion and/or stuffiness caused by colds, seasonal and other allergies, hay fever, sinus problems or allergic and non-allergic rhinitis, which may cause an increase in nasal discharge.

In certain embodiments the composition of the invention provides a therapeutically effective phenylephrine dose for at period of time after a single dose is administered to a subject. The subject can be any animal, human or otherwise, in need of treatment with phenylephrine. The period of time contemplated can be anywhere from 5 minutes to over 24 hours. It is contemplated that, by bypassing the first pass metabolism of the subject, a sustained therapeutic dosage can be obtained for a period of time from a single administration of the compositions of the invention that would be therapeutically equivalent to orally administered immediate release compositions that are typically administered in multiple dosages and absorbed through the gastrointestinal tract. Thus, when viewed in terms of pharmacokinetic parameters, a single administration of certain embodiments of the compositions of the invention will provide phenylephrine to the subject such that the subject exhibits a mean AUC and/or Cₘₐₓ of phenylephrine equivalent to from about 80% to about 125% of the AUC and/or Cₘₐₓ obtained by multiple doses of a standard immediate release oral dosage formulation of phenylephrine. Such standard immediate release oral dosage formulation of phenylephrine typically contain about 10 mg of phenylephrine and are administered in multiple doses, such as 2, 3, 4, 5, 6, or more doses, over a 24 hour period to provide for sustained therapeutic dosages.

Thus, certain embodiments of this invention provide a therapeutically effective phenylephrine dose for a period of time after a single dose is administered to a subject, wherein the period of time is at least about 5, 10, 15, 30, or 45 minutes, or at least about 1, 2, 3, 4, 5, 6, 7, 8, 9, 10, 11, 12, 13, 14, 15, 16, 17, 18, 19, 20, 21, 22, 23, or 24 hours. In addition, certain embodiments of the invention are formulated as a single dosage form to deliver phenylephrine or a pharmaceutically acceptable salt thereof to a subject in need thereof, such that the single dosage results in peak concentration of unmetabolized phenylephrine in plasma of the subject at a time point of from about 0.1 and about 1.5 hours after the composition contacts the oral mucosa. In certain embodiments of the invention, the amount of unmetabolized phenylephrine in the subject is maintained at a level greater than 20 picogram/ml. In certain embodiments of the invention, the amount of unmetabolized phenylephrine in the subject is maintained for a period of about one half to 12 hours after placing the composition in contact with the oral mucosa. The presence of unmetabolized phenylephrine is detectable by methods used by one skilled in the art for detecting pharmaceutical compounds in the plasma (P. Ptacek, et al. J. Chromatography B. 858 (2007), 263-268).

As used herein, the term “contacting of the oral mucosa” can comprise placing the composition of the invention under the tongue or on the floor of the mouth or in contact with the buccal mucosa. In certain embodiments of the invention, the compositions will contact the oral mucosa by means of placing a solid, semi-solid, or liquid form of the composition in the mouth. These methods of contacting may also include spraying the composition into the mouth in a manner that the composition is applied to the oral mucosa.

Thus, the invention further provides a method of systemically administering phenylephrine to a subject which comprises contacting oral mucosa with a pharmaceutical composition comprising phenylephrine or a pharmaceutically acceptable salt thereof, wherein the composition allows for absorption of phenylephrine by oral mucosa. In certain embodiments, the invention includes methods of treating symptoms of cold, influenza, or allergies in a subject in need thereof, comprising administering the pharmaceutical compositions described herein. In certain embodiments, the methods comprise administering the pharmaceutical composition every 8, 12, 16, or 24 hours.

In certain embodiments, the method of the invention comprises administering phenylephrine to the floor of the mouth underneath the tongue of the subject. In certain embodiments, the method of the invention comprises administering phenylephrine to the buccal mucosa of the subject.

### Phenylephrine Metabolite Activity Assays

The affinity and activity of phenylephrine metabolites were evaluated in human recombinant α₁ and α₂ adrenoreceptor binding and activity assays. PE undergoes extensive pre-systemic metabolism. After oral administration of approximately 24 mg of PE to healthy volunteers, four main metabolites were excreted in the urine (10). These metabolites are: 1) unconjugated m-hydroxymandelic acid (30% of dose); 2) sulfate conjugate of m-hydroxyphenylglycol; 3) sulfate conjugate of PE (47%); and 4) glucuronide conjugate of PE (12%). The purpose of the present studies was to determine the affinity and functional activity of m-hydroxymandelic acid, PE sulfate conjugate and PE glucuronide conjugate at the human recombinant α₁-adrenoreceptors (α₁ₐ and α_{1b} subtypes) and α₂-adrenoreceptors (α₂ₐ, α_{2b} and α_{2c} subtypes). Affinity of the metabolites was determined by receptor binding assays. Functional activity of the metabolites was assessed using an [³⁵S]-GTPγS binding exchange assay for the α₂ receptor subtypes and a cell-based calcium flux response for the α₁ receptor subtypes.

The major metabolites of PE were evaluated to determine their ability to bind to or activate the α₁ adrenoreceptor subtypes α₁ₐ and α_{1b} and the α₂ adrenoreceptor subtypes α₂ₐ, α_{2b} and α_{2c}. The metabolites evaluated were: 3-hydroxymandelic acid, PE sulfate and PE glucuronide. In each binding and functional assay the metabolites were compared to PE.

Materials And Methods

(R)-(−)-phenylephrine (PE), was obtained from Sigma (Cat. no. P6126-25G, CAS [61-76-7]). 3-hydroxymandelic acid, also known as m-hydroxymandelic acid, was obtained from Fluka (Cat no. 55520-1G. CAS [17119-15-2]), and characterized as described (11). (R)-PE sulfate was prepared as described from PE (11). By NMR (R)-PE-sulfate batch 4 was estimated to contain less than 0.1% PE (11). (R)-PE glucuronide was prepared as described (11). Two batches were prepared: batch 2 (“b2”) or batch 4 (“b4”). The amount of PE in the PE-glucuronide was estimated to be undetectable (b2) or ˜0.28% (b4) by LC/MS (11).

[³⁵S]-GTPγS Binding

Membranes (20 μg/well) from Chinese hamster ovary (CHO) cells expressing each of the α₂ adrenoreceptors were incubated for 30 minutes at room temperature with serial dilutions of phenylephrine (PE), PE metabolites or the standard, UK14304, or 1 μM cold GTPγS (non-specific binding) and 0.1 nM [³⁵S]-GTPγS in quadruplicate in NEN Basic FlashPlates®. Assay buffer was 75 mM Tris-HCl pH 7.4, 12.5 mM MgCl₂, 2 mM EDTA and 1 μM GDP. Plates were counted on a Packard TopCount. The percent increase over basal binding of [³⁵S]-GTPγS, a measure of efficacy, was calculated as follows: 100×[[mean total sample cpm−basal cpm]÷by basal cpm]. Basal cpm was defined as the mean cpm in the absence of agonist compound minus the mean non-specific binding cpm. Half-maximal effective concentrations (EC₅₀, concentration of compound required to give 50% of its own maximal stimulation) were calculated using nonlinear regression with GraphPad Prism.

Competition Binding Assays

Competition binding assays for the α₂ adrenoreceptors were performed using 20 μg membrane protein per well in binding buffer (75 mM Tris-HCl pH 7.4, 12.5 mM MgCl₂, 2 nM EDTA, 0.2% bovine serum albumin). [³H]-UK14304 was used as the radioligand. Competition binding for the α₁ adrenoreceptors was performed similarly with [³H]-Prazosin as the radioligand. The K_{d} of [³H]-UK14304 for α₂ₐ, α_{2b} and α_{2c} is: 0.9, 26.5 and 2.4 nM, respectively. The K_{d} of [³H]-Prazosin for α₁ₐ and α_{1b} is 0.2 and 0.3 nM, respectively. Competition binding was done using various concentrations of PE or PE metabolites as the cold competitor. Binding was terminated by rapid filtration through GF/C unifilter plates, presoaked with 0.3% polyethylenimine, with five washes with 0.5 ml cold 50 mM Tris-HCl ph 7.4, using a Packard Filtermate Harvester. After drying, bound radioactivity was determined by liquid scintillation counting (Packard TopCount) with Microscint 20, 50 μl/well. Binding data were analyzed using GraphPad Prism.

Cellular Calcium Flux

Intracellular calcium levels were measured using a fluorometric imaging plate reader (FLIPR). Cells expressing α₁ adrenoreceptors were cultured overnight at 15,000 cells/well in 96 well black-wall clear bottom plates (Packard). Adherent cells were loaded for 1 hour at 37° C. using the FLIPR Calcium Plus Assay Kit (Molecular Probes, Eugene, Oreg.), which included 2.5 mM probenecid (Sigma). Compounds (at 10 mM in 100% DMSO) were diluted in diluting buffer (HBSS, 20 mM HEPES, 2.5 mM probenecid, 0.5% BSA, pH 7.4). A titration of norepinephrine was included in every experiment and norepinephrine (at 1 μM) was also used as a plate standard on each assay plate. Cells were maintained at 37° C. throughout all calcium measurements. Fluorescence data was collected at 1 second interval for 60 seconds, followed by collection at 2 second intervals for 30 seconds. Background fluorescence was quantitated in wells containing cells with no additions and was subtracted from all experimental samples. All conditions were done in quadruplicate. Non-linear regression analysis using GraphPad Prism was used to calculate EC₅₀ values.

Data Analysis

PE was tested as a reference compound in all assays. Each metabolite was evaluated in each assay in at least 2 independent experiments and a representative assay of each metabolite/assay combination is shown. EC₅₀ and Kᵢ values are expressed as mean±SD of 24 independent assays.

A low level of PE was estimated to be present in the PE sulfate (less than 0.1%) or in PE glucuronide batch 4 (approximately 0.28%). Theoretical dose response curves were generated using nonlinear regression (Graphpad Prism) to estimate the activity expected if PE were present in the PE sulfate at 0.1% or in PE glucuronide batch 4 at 0.28%.

Results

The potency and affinity of PE and all PE metabolites tested are summarized in Table 1.

TABLE 1

| | | | | | | | | 3- | |
|---|---|---|---|---|---|---|---|---|---|
| | | | | | | PE | | hydroxymandelic | |
| | | PE | | PE sulfate | | glucuronide | | acid | |
| Receptor | Assay | Kᵢ | EC₅₀ | Kᵢ | EC₅₀ | Kᵢ | EC₅₀ | Kᵢ | EC₅₀ |
| a1a | Calcium | | 101 | | NA | | M | | NA |
| a1b | Calcium | | 14 | | NA | | M | | NA |
| a1a | Binding | 1873 | | NA | | NA | | NA | |
| a1b | Binding | 6737 | | NA | | NA | | NA | |
| a2a | GTPγS | | 225 | | NA | | NA | | NA |
| a2b | GTPγS | | 2334 | | NA | | NA | | NA |
| a2c | GTPγS | | 884 | | NA | | NA | | NA |
| a2a | Binding | 130 | | NA | | M | | NA | |
| a2b | Binding | 558 | | NA | | M | | NA | |
| a2c | Binding | 67 | | NA | | M | | NA | |

Numerical values represent mean Kᵢ or EC₅₀ nM
NA = Not Active
M = Not Active or Minimal activity in b4 consistent with PE contamination

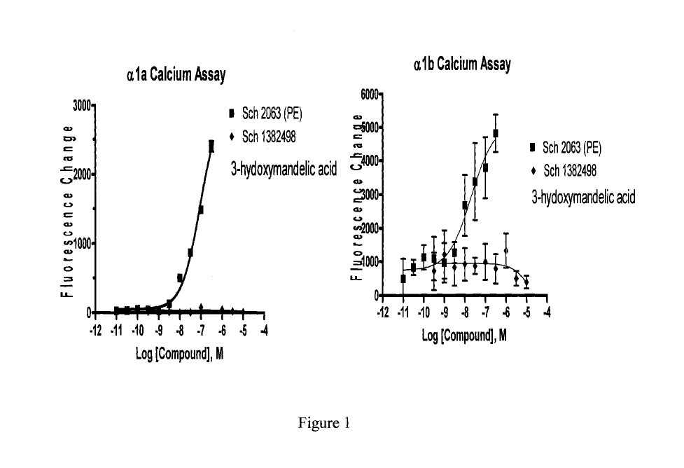

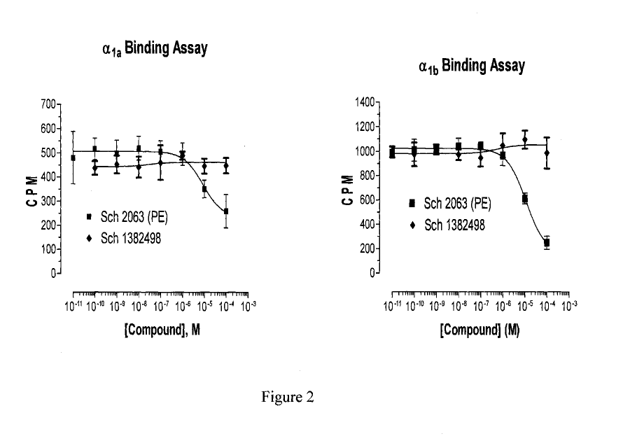

PE induced an increase in intracellular calcium in α₁ₐ-(EC₅₀=101±52 nM) and α_{1b}-expressing CHO cells (EC₅₀=13.6±20.6 nM). In contrast, 3-hydroxymandelic acid was not active in the α₁ₐ and α_{1b} calcium assays (FIG. 1). PE demonstrated binding to the α₁ₐ (Kᵢ=1873±1043 nM) and α_{1b} receptors (Kᵢ=6737±5650 nM). No appreciable binding to these receptors was detectable with 3-hydroxymandelic acid at concentrations up to 100 μM (FIG. 2).

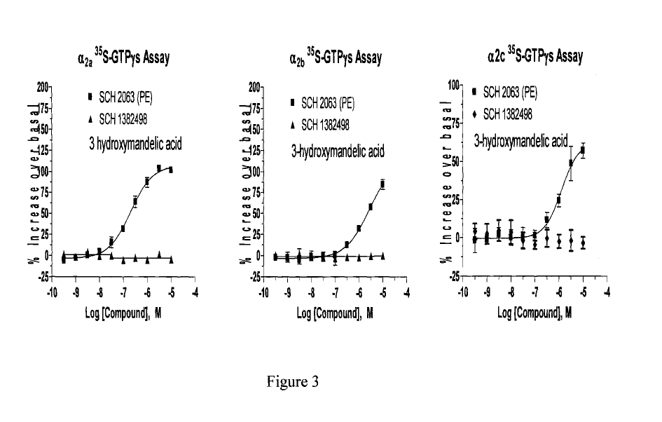

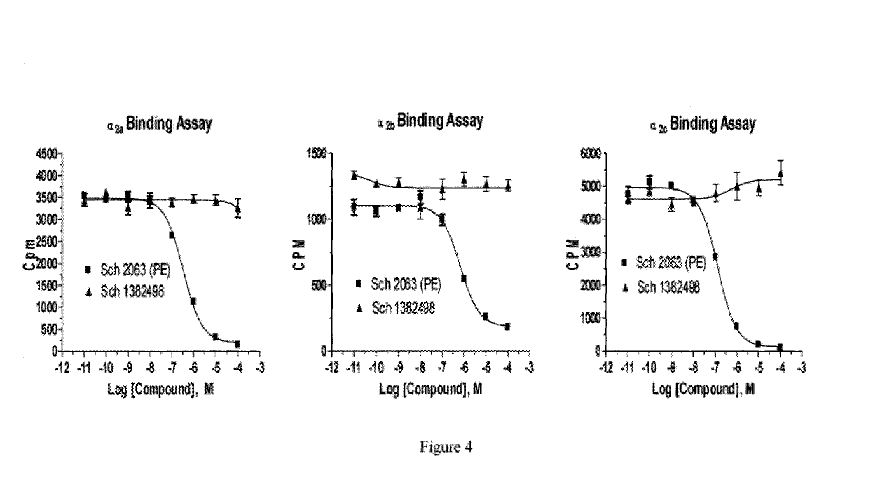

In an [³⁵S]-GTPγS binding exchange assay, PE demonstrated functional activity for the α₂ receptor subtypes. The potency of PE for the α₂ₐ, α_{2b} and α_{2c} subtypes is 225±46 nM, 2334±522 nM, and 884±312 nM, respectively. In contrast, 3-hydroxymandelic acid had no activity in the α₂ₐ, α_{2b} and α_{2c} ^{[35]}S-GTPγS assays (FIG. 3). Also, 3-hydroxymandelic acid demonstrated no significant binding to the α₂ receptor subtypes (FIG. 4). In contrast, PE bound to the α₂ₐ, α_{2b} and α_{2c} receptors with moderate affinity: Kᵢ=130±15 nM, 558±188 nM, and 67±16 nM, respectively.

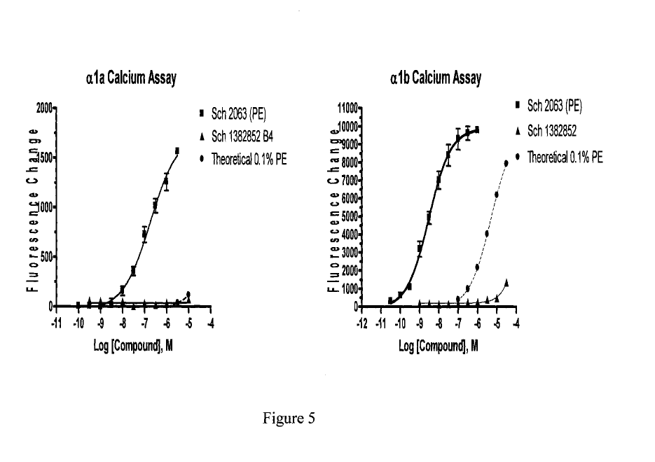

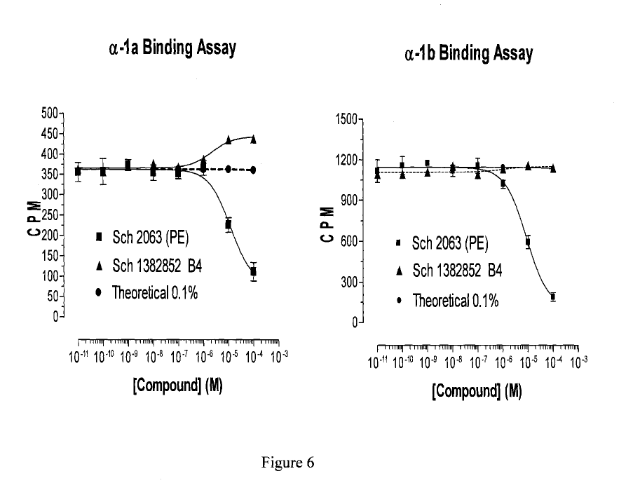

In contrast to PE, PE sulfate had no or minimal activity in the α₁ₐ or α_{1b} calcium assays, respectively (FIG. 5). Theoretical curves were also generated to indicate the activity expected if PE were present in PE sulfate at 0.1%, the limit of detection of PE by NMR. In both assays the activity of PE sulfate was much less than expected for PE if PE were present at the limit of assay detection (FIG. 5). No appreciable binding of PE sulfate was detected at the α₁ₐ and α_{1b} receptors (FIG. 6).

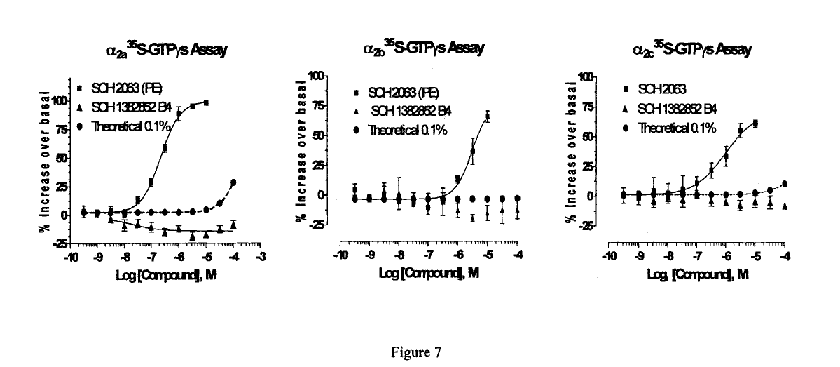

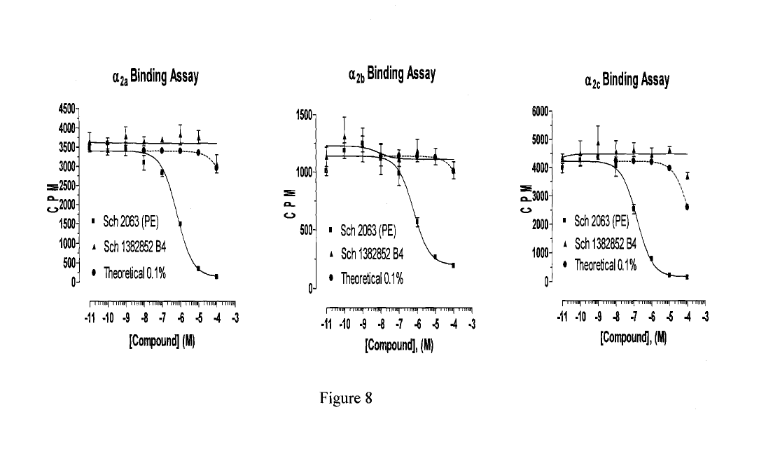

PE sulfate was also assessed for activity at the α₂ₐ, α_{2b} and α_{2c} subtypes using the [³⁵S]-GTPγS assays (FIG. 7). No activity of PE sulfate was detected and this was less than that expected for PE if PE were present at the limit of assay detection. In addition, no appreciable binding of PE sulfate was observed at the α₂ receptor subtypes (FIG. 8). The very minimal binding detected at 100 μM at each receptor subtype was less than that expected for PE if PE were present at the limit of assay detection.

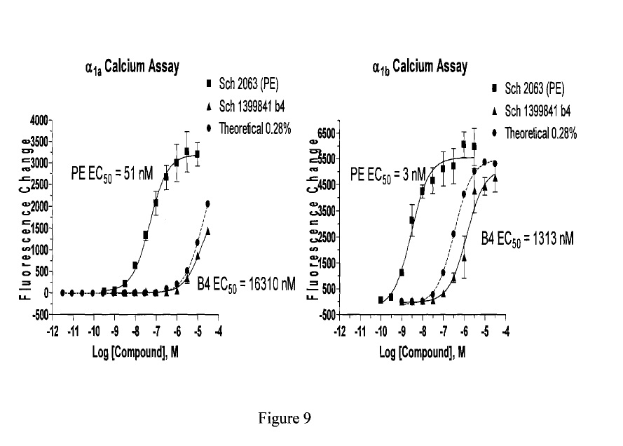

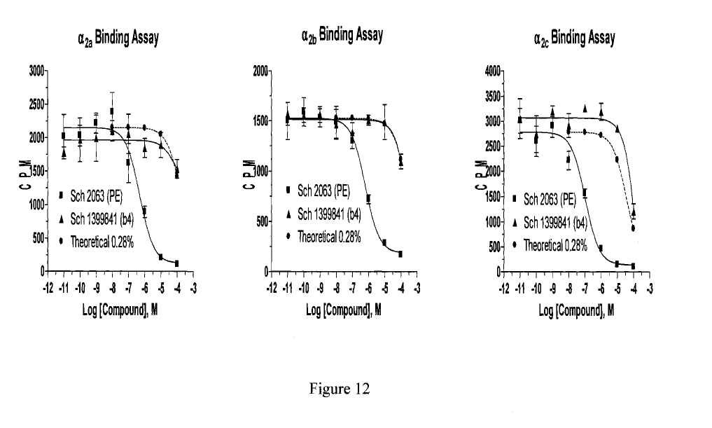

PE glucuronide was evaluated in the assays described above. PE glucuronide b4 was estimated to contain approximately 0.28% PE and was evaluated in the α₁ calcium assays (FIG. 9) and α₂ binding assays (FIG. 12). PE glucuronide b4 was ˜300-450-fold less potent than PE in inducing a calcium increase in the α₁ₐ or α_{1b} cells (FIG. 9). Theoretical curves were also generated to reflect the activity expected for contaminating PE which was present in PE glucuronide at approximately 0.28%. In both assays the activity of PE glucuronide was similar to or slightly less than that expected for PE if PE were present at 0.28% (FIG. 9). This indicates that the weak activity of PE glucuronide is attributable to the low level of contaminating PE.

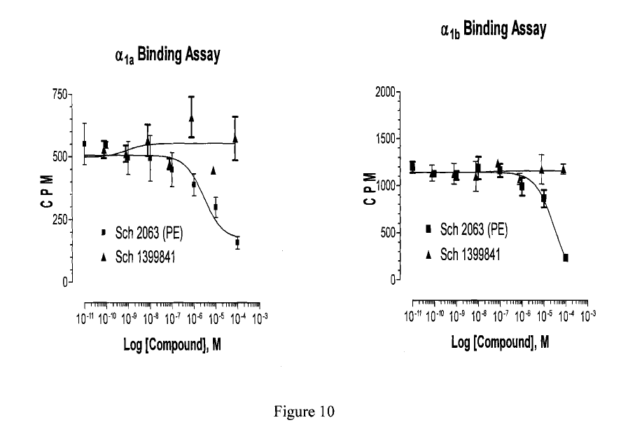

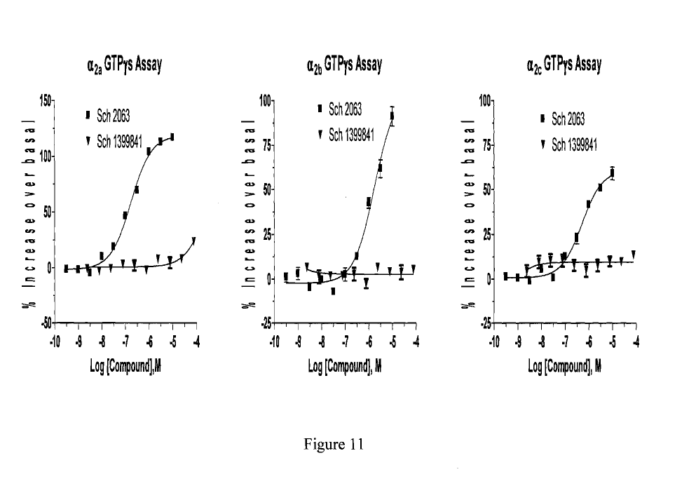

PE glucuronide b2, with no detectable PE, was evaluated in the α₁ binding assays (FIG. 10). No appreciable binding of PE glucuronide was detected at the α₁ₐ and α_{1b} receptors (FIG. 10). In the α₂ [³⁵S]-GTPγS assays (FIG. 11), PE glucuronide b2 stimulated very weak binding to α₂ₐ membranes only at the highest concentration tested, 100 μM. No stimulatory activity was observed in α_{2b} and α_{2c} membranes.

A small amount of binding of PE glucuronide b4 was observed at the α₂ receptor subtypes which was significantly less than that of PE (FIG. 12) and Kᵢ values could not be determined. Theoretical curves were generated to reflect the activity expected for contaminating PE which was present in PE glucuronide b4 at approximately 0.28%. In all α₂ receptor binding assays the activity of PE glucuronide b4 was similar to that expected for PE if PE were present at 0.28% (FIG. 12). This indicates that the weak activity of PE glucuronide is attributable to the low level of contaminating PE.

Conclusions

3-Hydroxymandelic acid had no activity at the highest concentration evaluated (10 μM) in the α₁ or α₂ assays assessing agonist activity. Both the calcium flux assay and the [³⁵S]-GTPγS binding exchange assay are considered sensitive assays of α₁ and α₂ adrenoreceptor activity, respectively, because each utilizes cells overexpressing the recombinant human adrenoreceptors. In addition, 3-hydroxymandelic acid had no affinity for the α₁ or α₂ receptor subtypes at the highest concentration evaluated (100 μM). Thus, 3-hydroxymandelic acid is an inactive metabolite of PE.

PE sulfate had no affinity for the α₁ or α₂ receptor subtypes at the highest concentration evaluated (100 μM). PE sulfate had no activity in the α₂ subtype [³⁵S]-GTPγS assays at the highest concentration evaluated (100 μM). A very low level of activity was detected in the α₁ calcium assays and this activity was much less than expected for PE if PE were present at the limit of assay detection. Thus, PE sulfate has minimal to no activity at the α₁ or α₂ adrenoreceptors. PE glucuronide was pharmacologically inactive in the α₁ and α₂ subtype receptor binding assays as well as in the assays measuring functional activity of the α₁ and α₂ receptors. PE glucuronide had no binding affinity for the α₁ₐ or α_{1b} receptors nor did it activate binding of [³⁵S]-GTPγS to the α₂ receptor subtypes. The minimal activity of PE glucuronide batch 4 observed in the α₁ₐ and α_{1b} calcium and α₂ receptor binding assays was completely consistent with the level of contaminating PE (0.28%).

### REFERENCES

1. Johannssen, V., S. Maune, J A Werner, H. Rudert, and A. Ziegler. 1997. Alpha 1-receptors at pre-capillary resistance vessels of the human nasal mucosa. Rhinology 35: 161-165.
2. Rizzo, C. A., L. M. Ruck, M. R. Corboz, S. P. Umland, Y. Wan, H. Shah, J. Jakway, L. Chen, K. McCormick, R. W. Egan, J. A. Hey. 2001. Postjunctional □_{2C}-adrenoceptor contractility in human saphenous vein. Eur. J. Pharmacol. 413: 263-269.
3. Corboz, M. R., L. M. Varty, C. A. Rizzo, J. C. Mutter, M. A. Rivelli, Y. Wan, S. Umland, H. Qiu, J. Jakway, K. D. McCormick, M. Berlin, and J. A. Hey. 2003. Pharmacological characterization of α₂-adrenoceptor-meidated responses in pig nasal mucosa. Autonomic and Autocoid Pharmacol. 23: 208-219.
4. Johnson, D A, and J. G. Hricik 1993. The pharmacology of alpha-adrenergic decongestants. 1993. Pharmacotherapy. 13: 110S-115S.
5. Watase, T. and M. Okuda. 1986. The effects of autonomotropic drugs on allergic nasal mucosa. Rhinology 24: 181-186.
6. McLeod, R., C. H. Erickson, G. G. Mingo, and J. A. Hey. 2001. Intranasal application of the α₂-adrenoceptor agonist BHT-920 produces decongestion in the cat. Am. J. Rhinol. 15: 407-415.
7. Chua, S. S., and S. I. Benrimoj. 1988. Non-prescription sympathomimetic agents and hypertension. Med. Toxicol. Adverse Drug Exp. 3: 387-417.
8. Ramey, J. T., E. Bailen, and R. F. Lockey. 2006. Rhinitis medicamentosa. J. Investig Allergol Clin Immunol. 16:148-155.
9. Graf, P.2005. Rhinitis medicamentosa: a review of causes and treatment. Respir. Med. 4: 21-29.
10. Bruce, R. B., and J. E. Pitts. 1968. The determination and excretion of phenylephrine in urine. Biochemical Pharmacology 17, 335-337.
11. R. Aslanian, C. Boyce, V. Hegde, M. de Lera Ruiz, M. Senior, Y. Yu, L.-K. Zhang. Synthesis and characterization of Phenylephrine metabolites. D-report #50144. Jul. 9, 2007.
12. P. Ptacek, J. Klima and J. Macek, Development and validation of a liquid chromatography-tandem mass spectrometry method for the determination of phenylephrine in human plasma and its application to a pharmacokinetic study. J Chromatography B. 858 (2007) 236-268.
13. Aungst, B. J., Rogers, N. J., and Shefter, E., Comparison of nasal, rectal, buccal, sublingual and intramuscular insulin efficacy and the effects of a bile salt absorption promoter, The J. Pharmacol. Exp. Ther., 244:23-27, 1988.
14. Yajaman S., Kuotsu K. and Bandyopadhyay A. K., Buccal bioadhesive drug delivery—A promising option for oarally less effective drugs, J. Controlled. Release. 114:2006, 15-40.
15. Rathbone, M. J. and Hadgraft, J., Absorption of drugs from the human oral cavity, Int. J. Pharm., 74:9-24, 1991.
16. Squier, C. A., The permeability of oral mucosa, Crit. Rev. Oral Biol. Med., 2:13-32, 1991.15. Squier, C
17. Squier, C. A. and Wertz, P. W. Structure and function of the oral mucosa and implications for drug delivery, in eds. M. J. Rathbone, Oral Mucosal Drug Delivery, Marcel Dekker, Inc., New York, NewYork, 1-26, 1996.
18. Galey, W. R., Lonsdale, H. K., and Nacht, S., The in vitro permeability of skin and buccal mucosa to selected drugs and tritiated water, J. Invest. Dermat., 67:713-717, 1976.
19. Sawicki W and Janicki S, Pharmacokinetics of verapamil and its metabolite norverapamil from a buccal drug formulation, Int J. Pharm., 238 (2002) 181-189
20. Janet A J. Hoogstraate and Philip W. Wertz, Drug Delivery Via the Buccal Mucosa, PSIT: 1(7) 1998: 309-313
21. Buket T., Yilmaz C., Olgun G., Sirri K., and Atilla H., Design and Evaluation of Sustain-release and Buccal adhesive propranolol Hydrochloride tablets, J. Controlled Release, 38 (1996): 11-20
22. Ehrnebo M, Boreus L O and Lonroth U., Bioavailability and first-pass metabolism of oral pentazocine in man, Clin Pharmacol Ther. 1977 December; 22(6):888-92.
23. Varsha A., and Mishra B., Design, Development, and Biopharmaceutical Properties of Buccoadhesive Compacts of Pentazocine, Drug, Dev. Inds. Pharm, 25(6)1999:701-709
24. Bruce, R. B. and Pifts, J. E. (1968) The Determination and excretion of phenylephrine in urine. Biochemcial Pharmacology 17, 335-337.
25. Bruce, R. B. and Pifts, J. E. (1968) The Determination and excretion of phenylephrine in urine. Biochemcial Pharmacology 17, 335-337.
26. Ibrahim, K. E. et al. (1983) The Mammalian Metabolism of R-(−)-m-Synephrine. Journal of Pharmacy and Pharmacology 35, 144-147.
27. Gumbhir, K. (1993) An Investigation of Pharmacokinetics of Phenylephrine and its Metabolites in Humans. In Pharmaceutical Sciences, pp. 216, University of Missouri-Kansas City.
28. Hengstmann, J. H. and Goronzy, J. (1982) Pharmacokinetics of ³H-Phenylephrine in Man. European Journal of Clinical Pharmacology 21, 335-341
29. Stockis, A. et al. (1995) Relative Bioavailability of Carbinoxamine and Phenylephrine from a Retard Capsule after Single and Repeated Dose Administration in Healthy Subjects. Arzneim.-Forsch./Drug Res. 45 (9), 1009-1012.
30. FDA. (2004) Proposed Rule—Docket No. 1976N-0052N/CP18. Cold, Cough, Allergy, Broncodilator, and Antiasthmatic Drug Products for Over-the-Counter Human Use; Proposed Ammendment of Monograph for Over-the-Counter Nasal Decongestant Drug Products. Federal Register 69 (211), 63482-63488.
31. Antonio, L. et al. (2003) Glucuronidation of catechols by human hepatic, gastric, and intestinal microsomal UDP-glucuronosyltransferases (UGT) and recombinant UGT1A6, UGT1A9, and UGT2B7. Archives of Biochemistry and Biophysics 411, 251-261.
32. Chen, G. et al. (2003) Human gastrointestinal sulfotransferases: identification and distribution. Toxicology and Applied Pharmacology 187, 186-197.
33. Doherty, M. M. and Charman, W. N. (2002) The Mucosa of the Small Intestine. How Clinically Relevant as an Organ of Drug Metabolism? Clinical Pharmacokinetics 41 (4), 235-253.
34. Gregory, P. A. et al. (2004) Regulation of UDP glucuronosyltransferases in the gastrointestinal tract. Toxicology and Applied Pharmacology 199, 354-363.
35. Teubner, W. et al. (2007) Identification and localization of soluble sulfotransferases in the human gastrointestinal tract. Biochemical Journal 404, 207-215
36. Radominska-Pandya, A. et al. (1998) UDP-glucuronosyltransferases in human intestinal mucosa. Biochemica et Biophysica Acta 1394, 199-208.
37. Pang, K. S. (2003) Modeling of Intestinal Drug Absorption: Roles of Transporters and Metabolic Enyzmes (For the Gillette Review Series). Drug Metabolism and Disposition 31 (12), 1507-1519.
38. Ileft, K. F. et al. (1990) Metabolism of Drugs and Other Xenobiotics in the Gut Lumen and Wall. Pharmcology and Therapeutics 46, 67-93.
39. Marathe, P. H. et al. (1995) Absorption and Presystemic Metabolism of Nefazodone Administered at Different Regions in the Gastrointestinal Tract of Humans. Pharmaceutical Research 12 (11), 1716-1721.
40. Kollar, C. et al. (2007) Meta-Analysis of the Efficacy of a Single Dose of Phenylephrine 10 mg Compared with Placebo in Adults with Acute Nasal Congestion Due to the Common Cold. Clinical Therapeutics 29 (6), 1057-1070.

The following examples describe certain embodiments of the compositions and methods of the invention. The examples are not intended, and should not be interpreted, to limit the scope of the invention which is more fully defined in the claims which appear thereafter.

### EXAMPLES

### Example 1

### Orally Disintegrating Tablet Dosage Forms

The following table shows a representative formulation for compositions of the invention in the form of an orally disintegrating tablet.

TABLE 2

| | Theoretical % | Amount per tablet |
|---|---|---|
| Ingredients | (w/w) | (mg) |
| Phenylephrine Hydrochloride | 1-30 | 1-45 |
| Mannitol | 30-60 | 45-90 |
| Crospovidone | 5-20 | 7.5-30 |
| Avicel PH 101 | 2-10 | 3-15 |
| Povidone | 1-3 | 1.5-4.5 |
| Magnesium stearate | 1 | 1.5 |
| Total | 100 | 150 |

The dosage forms are prepared by charging phenylephrine HCl, Avicel PH101, and Povidone to a granulator and mixing. The mixture is then granulated with water and passed through a screen, such as an 8 mesh screen. The granules are then dried, such as by using a tray dryer, and the dried granules are passed through a suitably-sized screening mill. The granulation is then mixed with selected excipients and pressed into tablets.

### Example 2

### Soft Gel Capsule Dosage Forms

The following table shows a representative formulation for compositions of the invention in the form of soft gel capsules.

TABLE 3

| | Theoretical % | |
|---|---|---|
| Ingredients | (w/w) | Amount (mg) |
| Phenylephrine Hydrochloride | 1-30 | 1-45 |
| PEG 400 | 10-50 | 15-75 |
| Water | 0-10 | 0-15 |
| Total | 100 | 150 |

The formulations are prepared by weighing PEG 400 and water and mixing well with a mixer. Phenylephrine HCl is then charged and mixed until all phenylephrine dissolved. The composition is then filled into softgel capsules.

### Example 3

### Buccal Tablet Dosage Forms

The following table shows a representative formulation for compositions of the invention in the form of buccal adhesive tablets having a diameter of approximately 7 mm and hardness 6-8 kP (kilopascal).

TABLE 4

| Ingredients | Theoretical % |
|---|---|
| Phenylephrine Hydrochloride | 1-50 |
| Carbopol ® 971P | 10-80 |
| Dextrose anhydrous | 5-50 |
| Corscarmellose Sodium | 0.5-15 |
| Magnesium Stearate | 0.1-1.0 |
| Flavor | 0.1-2 |
| Sucralose Micronized | 0.1-1 |
| Total | 100 |

The tablets are prepared by directly compressing a tablet mix containing between about 1 to about 75 mg of phenylephrine or pharmaceutically acceptable salt and about 90 to about 400 mg of excipients such as Carbopol® 971P as bioadhesive polymer, magnesium stearate as lubricant, corscarmellose sodium as supper disintegrate, granular sugar (e.g. dextrose, multidextrine, manitol etc.), sucralose as artificial sweetener and artificial flavors using a rotatory tablet press.

### Example 4

### Lozenge Dosage Forms

Lozenges are flavored dosage delivery systems for medication that are held in the mouth, wetted with saliva and sucked until dissolution occurs. A lozenge that dissolves slower is more preferable to allow for most of the drug to be absorbed from the buccal cavity and less swallowed and lost in the GI tract. The following table shows a representative formulation for compositions of the invention in the form of lozenges having a diameter of approximately 20 mm and hardness of between about 12 and about 30 kP.

TABLE 5

| | Theoretical % |
|---|---|
| Ingredients | (w/w) |
| Phenylephrine Hydrochloride | 1-50 |
| Carbopol 971P | 5-40 |
| Xanthan Gum | 5-30 |
| Mannitol | 10-70 |
| Magnesium Stearate | 0.1-1 |
| Flavor | 0.1-2 |
| Sweetener | 0.1-2 |
| Total | 100 |

The lozenges are prepared by direct compressing a tablet mix consisting of 5-75 mg of phenylephrine and 80-900 mg of suitable excipients such as magnesium stearate, mannitol, carbopol 971P and xanthan gum using a rotatory tablet press.

### Examples 5-8

### Buccal/Sublingual Film Dosage Form

The following table shows representative formulations for compositions of the invention in the form of rapidly disintegrating/dissolving films for oral consumption with no mucoadhesion.

TABLE 6

| | Example 5 | | Example 6 | |
|---|---|---|---|---|
| | Amount | Percent | Amount | Percent |
| Ingredients | (mg/film) | (%) | (mg/film) | (%) |
| Phenylephrine HCl | 10.00 | 28.30 | 20.00 | 33.83 |
| Pullulan | 14.12 | 39.97 | 30.00 | 50.75 |
| Xanthan Gum | 0.08 | 0.23 | 0.08 | 0.14 |
| Locust Bean Gum | 0.10 | 0.28 | 0.10 | 0.17 |
| Carrageenan | 0.41 | 1.16 | 0.41 | 0.70 |
| Sodium Benzoate | 0.10 | 0.28 | 0.10 | 0.17 |
| Acesulfame Potassium | 0.68 | 1.92 | 0.68 | 1.15 |
| Aspartame NF | 1.91 | 5.41 | 1.91 | 3.23 |
| Purifed Water USP/EP | * | * | * | * |
| Cooling Agent | 0.14 | 0.39 | 0.14 | 0.24 |
| Menthol | 2.73 | 7.73 | 2.73 | 4.62 |
| Polysorbate 80 NF | 0.48 | 1.36 | 0.48 | 0.81 |
| Atmos 300 | 0.48 | 1.36 | 0.48 | 0.81 |
| Propylene Glycol | 4.10 | 11.60 | 2.00 | 3.38 |
| Total Dose Weight | 35.33 | 100 | 59.11 | 100 |

* Calculated assuming complete evaporation of water from the films after drying. Enough water is used to enable efficient processing.

Ingredient ranges for one film dose according to this aspect of the invention can be as follows:

TABLE 7

| | Theoretical % |
|---|---|
| Ingredient | (w/w) |
| Phenylephrine HCl or | 1-35 (5-20 mg) |
| similar salt | |
| Pullulan | 40-80 |
| Xanthan Gum | 0.1-0.5 |
| Locust Bean Gum | 0.1-0.5 |
| Carrageenan | 0.70-2 |
| Sodium Benzoate | 0.1-0.4 |
| Acesulfame Potassium | 1-3 |
| Aspartame | 3-7 |
| Polysorbate 80 | 0.8-2 |
| Atmos 300 | 0.8-2 |
| Propylene Glycol | 3-20 |

The films in Examples 5 and 6 are prepared as follows. The film-forming ingredients (e.g. pullulan, xanthan gum, locust bean gum, and carrageenan) other than Polysorbate 80 and Atmos 300 are mixed and hydrated in hot purified water to form a gel and stored in a refrigerator overnight at a temperature of approximately 4° C. to form Preparation A. The sweetener and Phenylephrine Hydrochloride are dissolved in purified water to form Preparation B. Preparation B is added to Preparation A and mixed together to form Preparation C. The flavoring agents (e.g. cooling agent and menthol) are mixed to form Preparation D. The Polysorbate 80 and Atmos 300 are added to Preparation D and mixed well to form Preparation E which is added to Preparation C and mixed well to form Preparation F. Preparation F is Poured on a Mold and Cast to Form a Film of desired thickness at room temperature. The film is dried using warm air and cut into desired dimensions, packaged and stored. The films will have a very rapid dissolving time, on the order of about 10 seconds.

The following table shows representative formulations for compositions of the invention in the form of disintegrating/dissolving films for oral consumption with mucoadhesive properties:

TABLE 8

| | Example 7 | | Example 8 | |
|---|---|---|---|---|
| | Amount | Percent | Amount | Percent |
| Ingredients | (mg/film) | (%) | (mg/film) | (%) |
| Phenylephrine HCl | 10.00 | 14.22 | 20.00 | 21.62 |
| Sorbitol | 3.00 | 4.27 | 5.00 | 5.41 |
| Polyplasdone (Kollidon | 1.50 | 2.13 | 2.50 | 2.70 |
| 30) | | | | |
| Glycerol | 5.00 | 7.11 | 5.00 | 5.41 |
| Propylene Glycol | 5.00 | 7.11 | 5.00 | 5.41 |
| Polysorbate 80 NF | 4.00 | 5.69 | 6.00 | 6.49 |
| Polyoxyethylene (23) | 8.00 | 11.38 | 10.00 | 10.81 |
| lauryl ether (Brij 35) | | | | |
| Peppermint Flavor | 5.00 | 7.11 | 7.50 | 8.11 |
| Aspartame | 0.80 | 1.14 | 1.50 | 1.62 |
| Hydroxypropylmethyl | 28.00 | 39.83 | 30.00 | 32.43 |
| cellulose | | | | |
| Purfied Water USP/EP | * | * | * | * |
| Ethanol USP | * | * | * | * |
| Total Dose Weight | 70.30 | 100 | 92.50 | 100 |

* Calculated assuming complete evaporation of water and ethanol from the films after drying. Enough water and ethanol is used to enable efficient processing. A preservative, e.g. sodium benzoate, can be added as an anti-microbial agent.

Ingredient ranges for one film dose according to this aspect of the invention can be as follows:

TABLE 9

| | Theoretical % |
|---|---|
| Ingredient | (w/w) |
| Phenylephrine HCl or | 1-25 (1 to 20 mg) |
| similar salt | |
| Sorbitol | 1-5 |
| Kollidon 30 | 1-3 |
| Glycerol | 1-10 |
| Propylene Glycol | 1-10 |
| Sodium Benzoate | 0.1-1 |
| Aspartame | 1-5 |
| Polysorbate 80 | 1-7 |
| Brij 35 | 5-12 |
| Propylene Glycol | 1-10 |
| Hydroxypropylmethyl | 20-40 |
| cellulose | |

The films in Examples 7 and 8 are prepared as follows. Sorbitol, Kollidon 30, glycerol, propylene glycol, polysorbate 80, Brij 35, peppermint flavor and aspartame are dissolved in a sufficient amount of water and ethanol (e.g. 800 gram for an approximate batch size of 75 gram) at 60° C. while stirring. After all the ingredients are dissolved (clear solution is obtained), add hydroxypropylmethyl cellulose (HPMC) while stirring. After the HPMC is completely dissolved, the solution is cooled to room temperature and coated onto a suitable carrier web (e.g. non-siliconized, polyethylene-coated kraft paper) using conventional coating and drying conditions. Coating gap and web speed have to be adjusted to achieve a dry film thickness between 20 and 50 micron. The resulting film is peeled off the carrier web and cut into pieces of a suitable shape and size.

### Example 9

### Semi-Solid (Chewing Gum) Dosage Forms

The following table shows a representative formulation for compositions of the invention in the form of a semi solid chewing gum composition:

TABLE 10

| Ingredients | Theoretical % |
|---|---|
| Phenylephrine Hydrochloride | 1-50 |
| Gum base | 20-80 |
| Menthol | 0.1-1.0 |
| Flavor | 0.1-10 |
| Sweetener | 0.1-5 |
| Total | 100 |

The chewing gum compositions are comprised of a water insoluble chewing gum base portion, a water soluble portion includes sweeteners and phenylephrine or its pharmaceutically acceptable salt, fillers that may be insoluble or partially soluble and flavors and colorants. Phenylephrine and all soluble ingredients except filler are dissolved in a mixing vessel and granulated with the fillers. The granulation is dried in a suitable dryer and then milled with suitable particle size distributions. The milled granulation is then mixed with gum base in a suitable mixer. The mix is then compressed into chewing gum using suitable roll compression equipment.

What is claimed is:

1. A pharmaceutical composition comprising phenylephrine or a pharmaceutically acceptable salt thereof, wherein the composition is formulated to be applied to oral mucosa to allow for enhanced systemic absorption of therapeutically active form of phenylephrine.

2. The composition of claim 1 wherein the composition is formulated to allow for immediate systemic absorption of therapeutically active form of phenylephrine.

3. The composition of claim 1 wherein the composition is formulated to allow for sustained systemic absorption of therapeutically active form of phenylephrine.

4. A pharmaceutical composition suitable for sublingual systemic administration of phenylephrine or a pharmaceutically acceptable salt thereof, wherein the composition allows for systemic absorption of phenylephrine from the floor of the mouth.

5. The composition of claim 4 wherein the composition is formulated to provide an immediate release of phenylephrine.

6. The composition of claim 4 wherein the composition is formulated to provide a sustained release of phenylephrine.

7. A pharmaceutical composition suitable for buccal systemic administration of phenylephrine or a pharmaceutically acceptable salt thereof, wherein the composition allows for absorption of phenylephrine from the buccal mucosa.

8. The composition of claim 7 wherein the composition is formulated to provide an immediate release of phenylephrine.

9. The composition of claim 7 wherein the composition is formulated to provide a sustained release of phenylephrine.

10. The composition of claim 1 wherein the composition provides a release of phenylephrine to provide a measurable plasma concentration of therapeutically active form of phenylephrine in the subject for a period of at least four hours.

11. The composition of claim 1 wherein the composition provides a release of phenylephrine to provide a measurable plasma concentration of therapeutically active form of phenylephrine in the subject for a period of at least six hours.

12. The composition of claim 1 wherein the composition provides a release of phenylephrine to provide a measurable plasma concentration of therapeutically active form of phenylephrine in the subject for a period of at least eight hours.

13. The composition of claim 1 wherein the composition provides a release of phenylephrine to provide a measurable plasma concentration of therapeutically active form of phenylephrine in the subject for a period of at least twelve hours.

14. The composition of claim 1 wherein the composition provides a release of phenylephrine to provide a measurable plasma concentration of therapeutically active form of phenylephrine in the subject for a period of at least sixteen hours.

15. The composition of claim 1 wherein the composition provides a release of phenylephrine to provide a measurable plasma concentration of therapeutically active form of phenylephrine in the subject for a period of at least twenty hours.

16. The composition of claim 1 wherein the composition provides a release of phenylephrine to provide a measurable plasma concentration of therapeutically active form of phenylephrine in the subject for a period of at least twenty four hours.

17. A method of systemically administering phenylephrine which comprises contacting oral mucosa with a pharmaceutical composition comprising phenylephrine or a pharmaceutically acceptable salt thereof, wherein the composition allows for release of phenylephrine to oral mucosa.

18. A dissolvable composition comprising phenylephrine distributed within an aqueous soluble base material, wherein the composition is provided as a strip for inter-oral administration of phenylephrine to the mucus membranes of the mouth of a human or animal subject.

19. The composition of claim 18 wherein base material comprises a carrier which is conformed as a strip to serve as a delivery system for a measured dose of phenylephrine.

20. The composition of claim 18 wherein the strip comprises phenylephrine coated on the strip.

21. The composition of claim 18 wherein the strip comprises a flexible film having a thickness of from about 20 microns to about 250 microns.

22. The composition according to claim 18 wherein the carrier or base material of the strip comprises a soluble gel material.

23. The composition according to claim 1 wherein a part or all of the phenylephrine or pharmaceutically acceptable salt thereof are encapsulated within encapsulation structures.

24. The composition according to claim 23 wherein the encapsulation structures are selected to adhere to the mucous membranes of the oral cavity.

25. The composition according to claim 1 wherein the encapsulation structures are selected to release the phenylephrine or pharmaceutically acceptable salt thereof slowly over time.

26. The composition according to claim 23 wherein the encapsulation structures comprise multilamellar microparticles.

27. A bioerodible, water-soluble, carrier device comprising a non-bioadhesive backing layer, a bioadhesive layer and a composition comprising phenylephrine or a pharmaceutically acceptable salt thereof, wherein the bioadhesive layer is formulated to adhere to a mucosal surface of a mammal and provides sustained delivery of the composition.

28. The carrier device of claim 27 wherein the composition further comprises a fluid carrier suitable for administration to a mucosal surface of a mammal.

29. The carrier device of claim 28 wherein the fluid carrier comprises acetic acid, acetone, anisole, 1-butanol, 2-butanol, butyl acetate, tert-butylmethyl ether, cumene, dimethyl sulfoxide, ethanol, ethyl acetate, ethyl ether, methanol, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, or tetrahydrofuran.

30. The carrier device of claim 27 wherein the composition further comprises a polymeric or nonpolymeric hydrophilic agent.

31. The carrier device of claim 30 wherein the hydrophilic agent comprises polyethylene glycol.

32. The carrier device of claim 27 wherein the bioadhesive layer is water-soluble.

33. The carrier device of claim 27 wherein the bioadhesive layer comprises a film forming water-soluble polymer.

34. The carrier device of claim 27 wherein the bioadhesive layer comprises a bioadhesive polymer.

35. The carrier device of claim 33 wherein the film forming water soluble polymer of the bioadhesive layer comprises hydroxyethyl cellulose, hydroxypropyl cellulose, hydroxypropylmethyl cellulose, hydroxyethylmethyl cellulose, or a combination thereof.

36. The carrier device of claim 33 wherein the film forming water soluble polymer of the bioadhesive layer is crosslinked or plasticized.

37. The carrier device of claim 34 wherein the bioadhesive polymer of the bioadhesive layer comprises polyacrylic acid, sodium carboxymethyl cellulose or polyvinylpyrrolidone or a combination thereof.

38. The carrier device of claim 37 wherein the polyacrylic acid is partially crosslinked.

39. The carrier device of claim 27 wherein the non-bioadhesive backing layer comprises a pharmaceutically acceptable, film-forming, water-soluble polymer.

40. The carrier device of claim 39 wherein the pharmaceutically acceptable, film-forming, water-soluble polymer is hydroxyethyl cellulose, hydroxypropyl cellulose, hydroxypropylmethyl cellulose, hydroxyethylmethyl cellulose, polyvinyl alcohol, polyethylene glycol, polyethylene oxide, ethylene oxide-propylene oxide co-polymers, or a combination thereof.

41. A composition for buccal or sublingual application comprising a distribution of multilayer microparticles in a base, wherein phenylephrine or a pharmaceutically acceptable salt thereof is adsorbed within the layers of the microparticles so as to be progressively released over time to the buccal or sublingual mucosa.

42. The composition of claim 41 in the form for application by means of a spray, mousse or drench.

43. The composition of claim 41 comprising a distribution of multilayer microparticles in a soluble solid or gel base, the base material being formulated to dissolve within the mouth and liberate the microparticles to allow for contact of the microparticles with the mucous membranes of the oral cavity.

44. Composition of claim 41 wherein multilayer microparticles are selected to exhibit good adhesion to the mucous membranes of the oral cavity.

45. The composition of claim 41 wherein the multilayer microparticles are in the range 0.1-10 microns.

46. The composition of claim 41 wherein the multilayer microparticles comprise an aerosolized spray.

47. The composition according to claim 41 wherein the microparticles generally comprise polar structures with a positive surface charge.

48. The composition according to claim 1 further comprising additional active ingredients chosen from the group consisting of antihistamines, antibacterials, anti-inflammatory agents, and analgesic compounds.

49. The composition of claim 48 wherein the antihistamine is chosen from the group consisting of dipherihydramine, chlorpheniramine, tripelennamine, promethazine, clemastine, doxylamine, astemizole, terfenadine, loratadine, desloratadine, cimetadine, famotidine, nizatidine, ranitidine, cromolyn and combinations thereof.

50. Composition according to claim 1 further comprising one or more lubricating and/or moisturising oils.

51. The composition of claim 50 wherein the lubricating and/or moisturising oils are selected from the group consisting of hyaluronic acid or sodium hyaluronique, glycerol, calendula officinalis flower extract or glycerin extract, guar hydroxypropyltrimonium chloride, xanthan gum, cellulose gum, sodium chloride, olive oil, sunflower oil, almond oil, sesame oil, aloe vera, aloe barbadensis, and combinations thereof.

52. A drug delivery device adapted for application sublingually of the oral cavity for fast release thereon of a composition comprising phenylephrine or a pharmaceutically acceptable salt thereof, said device comprising a body having the composition distributed therein and having a size and shape suitable for sublingual application

53. The device of claim 52 wherein the body is in the form of a tablet, a softgel capsule, a fast dissolving film.

54. The devise of claim 53 wherein the tablet is a fast dissolving or fast melting tablet.

55. A pharmaceutical formulation adapted for application and adherence to the mucosa of the oral cavity for sustained release thereon of a composition comprising phenylephrine or a pharmaceutically acceptable salt thereof wherein the composition is in the form of a liquid or semisolid.

56. The pharmaceutical formulation of claim 55 wherein the liquid or semisolid congeals after application to oral mucosa.
